(19) United States
(12) Patent Application Publication
(10) Pub. No.: US 2010/0099602 A1
(43) Pub. Date: Apr. 22, 2010

(54) PHARMACEUTICAL COMPOSITIONS AND METHODS OF USE FOR THE PREVENTION AND TREATMENT OF HYPOXIC INJURY

(75) Inventors: Peter Petzelbauer, Wien (AT); Rainer Henning, Uetliburg (CH)

Correspondence Address: HOFFMANN & BARON, LLP, 6900 JERICHO TURNPIKE, SYOSSET, NY, 11791, US

(73) Assignee: FIBREX MEDICAL RESEARCH & DEVELOPMENT GMBH, Wien (AT)

(21) Appl. No.: 12/580,049

(22) Filed: Oct. 15, 2009

Related U.S. Application Data
(60) Provisional application No. 61/105,738, filed on Oct. 15, 2008.

(51) Int. Cl. A61K 38/14; A61K 38/10; A61K 38/16; A61K 38/13; A61P 29/00

(52) U.S. Cl. 514/8; 514/14; 514/12; 514/11

(57) ABSTRACT

The invention is directed to compositions and methods for using the same for the prevention and treatment of hypoxic conditions, ischemia/reperfusion injury and the sequels thereof.

### CROSS REFERENCE TO RELATED APPLICATION

This application claims benefit to of U.S. provisional application 61/105,738 filed Oct. 15, 2008.

### FIELD OF THE INVENTION

The present invention relates generally to compositions and methods for using pharmaceutical preparations for the prevention and treatment of hypoxic conditions, ischemia/reperfusion injury and the sequels thereof.

### BACKGROUND OF THE INVENTION

Reperfusion injury refers to damage to tissue, vasculature, organs or to the whole warm-blooded organism that is deprived of sufficient oxygen supply from blood and resulting in ischemia. Subsequently, reperfusion by blood further damages the tissue, vasculature and organ(s). The absence of oxygen and nutrients from blood create a condition in which the return of circulation results in inflammation and other damage. Reperfusion injury is due in part to the inflammatory response of damaged tissues to the ischemic insult. Leukocytes (white blood cells) from circulation release inflammatory mediators (i.e. interleukins) and free radicals. A number of physiological and pathophysiological processes are involved in the advent of reperfusion injury.

Recently, peptides and peptide analogs were described that prevent acute inflammation and vascular leak by binding to vascular endothelial (VE)-cadherin (WO 02/48180, WO 07/95659, WO 07195660, and WO 7/95661). It was shown that a peptide matching amino acids 15-42 of the Bbeta chain of fibrin blocks binding of fibrin fragments to endothelial surfaces and blocks inflammation in vitro, prevents myocardial inflammation and reduces myocardial infarct following ischemia/reperfusion injury (WO 02/48180).

Furthermore, it has been shown that compounds preventing the opening of the mitochondrial permeability transition pore (MPTP) and activating the reperfusion injury salvage kinase (RISK) pathway have a protective effect on reperfusion injury (D. M. Yellon and D. J. Hausenloy, New Engl. J. Med. (2007)). Compounds showing this kind of biological activity include for instance cyclosporine, Sanglifehrin A (SJ Clarke et al., J. Biol. Chem., (2002)), NIM811 (L. Argaud et al., Circulation (2005)), glucagon-like peptide-1 (AK Bose et al., Diabetes, (2005)), erythropoietin (AJ Bullard et al., Basic Res. Cardiol., (2005)), atorvastatin (R M Bell, D M Yellon, J. Amer. Coll. Cardiol. (2003)) and atrial natriuretic peptide (X M Yang et al., Basic Res. Cardiol. (2006)).

Accordingly, there is a need for compositions and methods that prevent or treat hypoxic conditions and particularly ischemia-reperfusion injury through novel means.

### SUMMARY OF INVENTION

The present invention is directed to compositions and methods useful to prevent, reduce and treat hypoxic injury.

In one aspect of the invention, there is provided compositions comprising:

(i) compound (I):
H₂N-GHRPX₁X₂X₃-β-X₄X₅X₆X₇X₈X₉X₁₀-X₁₁, or a physiologically acceptable salt thereof, wherein
X₁-X₁₀ denote one of the 20 genetically coded amino acids or wherein X₂, X₃, X₆, X₇, X₈, X₉ and X₁₀ individually or jointly denote a single chemical bond
X₁₁ denotes OR₁ wherein R₁ is hydrogen or (C₁-C₁₀) alkyl, NR₂R₃ with R₂ and R₃ identical or different and denote hydrogen, (C₁-C₁₀) alkyl; —W-PEG_{S-60K}, wherein PEG is attached via a spacer W to the N-atom of NR₂R₃; or NH—Y-Z-PEG_{5-60K}, wherein Y denotes a single chemical bond or a genetically coded amino acids from the group S, C, K or R and wherein Z denotes a spacer, via which PEG can be attached;
and
β denotes a genetically coded amino acid, a non-naturally occurring amino acid or a peptidomimetic element selected from the following: L-proline, D-proline, L-hydroxyproline, D-hydroxyproline, L-(O-benzyl)-hydroxyproline, D-(O-benzyl)-hydroxyproline, L-(O-tert. butyl)-hydroxyproline, 4-(O-2-naphtyl)-hydroxyproline, 4-(O)-2-naphtyl-methyl)-hydroxyproline, 4-(O-phenyl)-hydroxyproline, 4-(4-phenyl-benzyl)-proline, cis-3-phenyl-proline, cis-4-phenyl-proline, trans-4-phenyl-proline, cis-5-phenyl-proline, trans-5-phenyl-proline, 4-benzyl-proline, 4-bromobenzyl-proline, 4-cyclohexyl-proline, 4-fluor-proline, L-tetrahydroisoquinoline-2-carboxylic acid (L-Tic), a diastereomers of octahydro-indole-2-carboxylic acid (Oic), a diastereomers of 1-aza-bicyclo[3,3,0]octane-2-carboxylic acid,

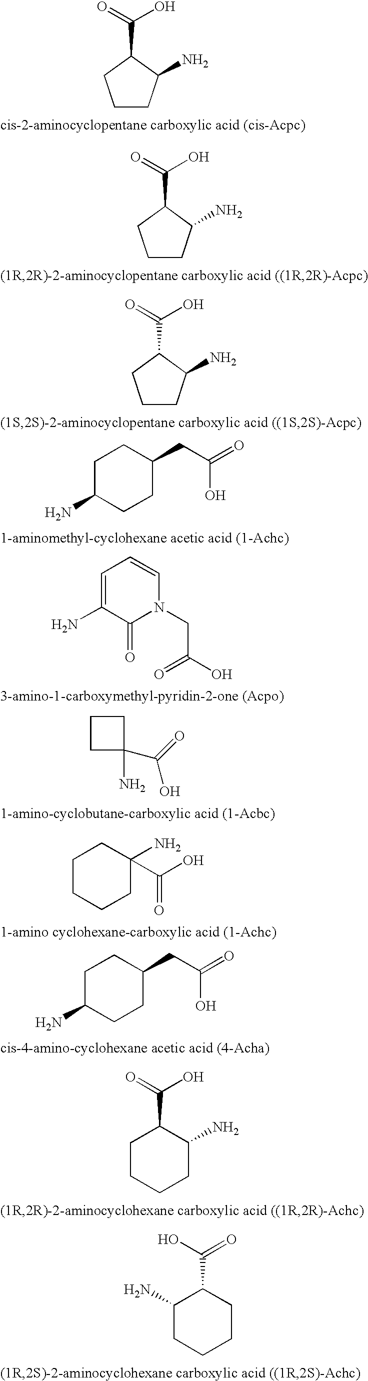

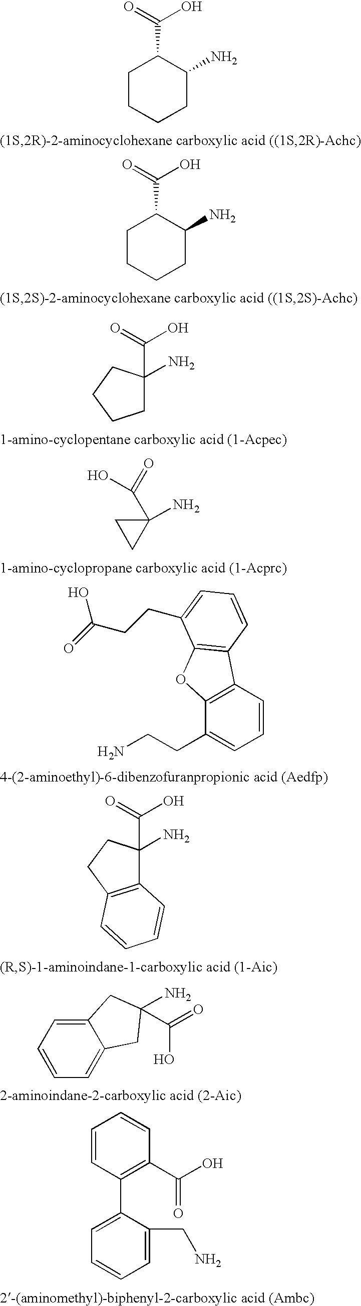

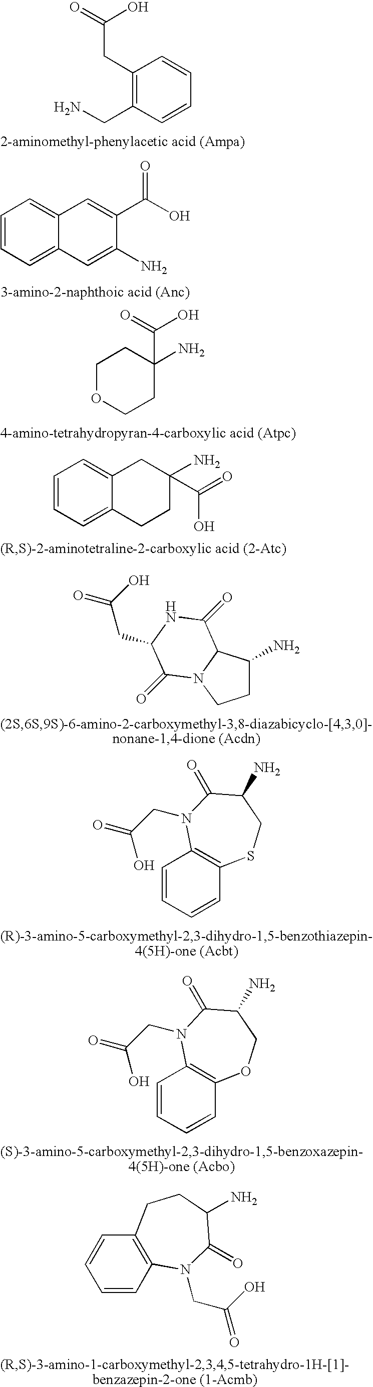

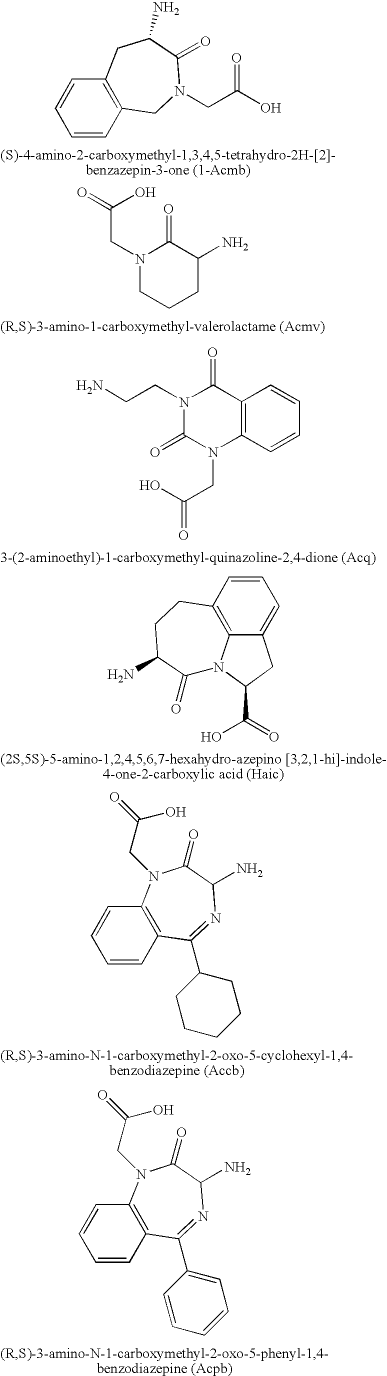

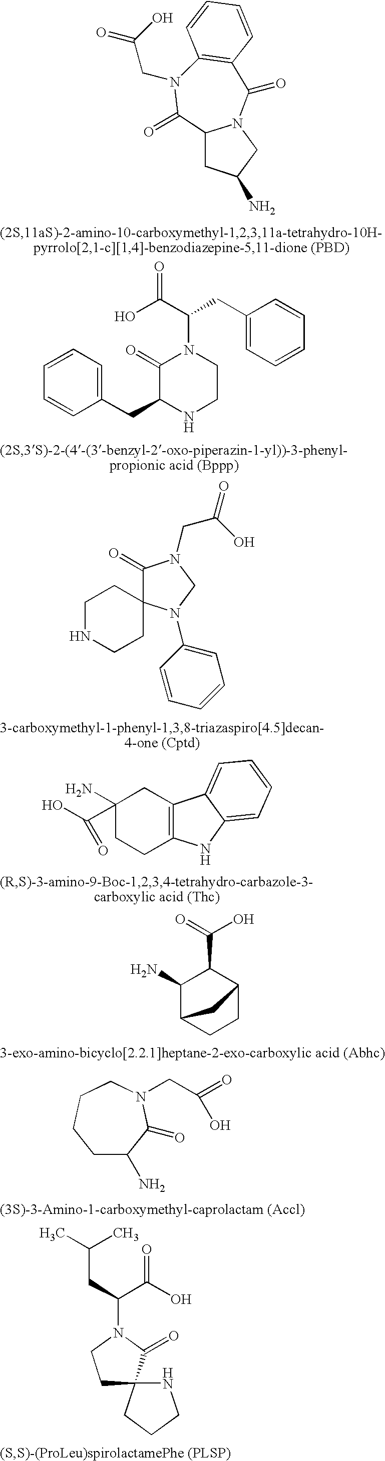

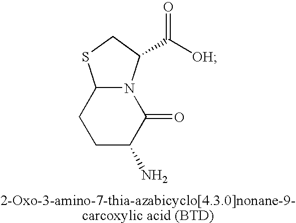

and
(ii) at least one other compound wherein the other compound activates the reperfusion injury salvage kinase pathway or inhibits the mitochondrial permeability transition pore.

In another aspect of the invention, there is provided compositions comprising:

(i) compound A

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg

or a salt thereof, wherein the amino terminus is

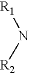

wherein R₁ and R₂ are either the same or different, and wherein R₁ and R₂ are each selected from the group consisting of hydrogen and a saturated or unsaturated hydrocarbon residue, said residue having from 1 to 10 carbon atoms;
   and
   (ii) at least one other compound wherein the other compound activates the reperfusion injury salvage kinase pathway or inhibits the mitochondrial permeability transition pore.

In yet another aspect of the invention, there is provided compositions comprising:

(i) compound B
H₂N-GHRPLDKKREEAPSLRPAPPPISGGGYR-X₁₇ or a physiologically acceptable salt thereof, wherein:
X₁₇ denotes NR₃R₄ or C(NR₃R₄)—(S-succinimido)-(polyethylene glycol (PEG)_{5-40K}), wherein the succinimide is linked to the sulfur atom of the cysteine residue via C-atom 3, R₃ and R₄ being identical or different and being hydrogen or (C₁-C₁₀)-alkyl;
   and
   (ii) at least one other compound wherein the other compound activates the reperfusion injury salvage kinase pathway or inhibits the mitochondrial permeability transition pore.

In still another aspect of the invention, there is provided compositions comprising:

(i) at least one compound selected from:
compound C

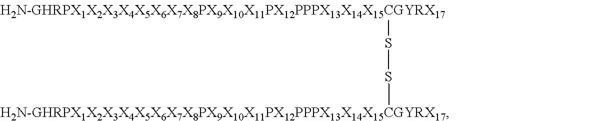

or
compound D

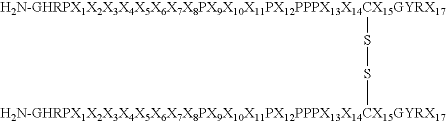

or a physiologically acceptable salt thereof, wherein:
X₁-X₁₅ denote one of the 20 genetically encoded amino acids,
X₁₇ denotes a residue OR₅, wherein R₅ is hydrogen or (C₁-C₁₀)-alkyl; NR₆R₇,
wherein R₆ and R₇ are identical or different and denote hydrogen or (C₁-C₁₀-alkyl; -PEG_{5-60K}-CO—NR₆R₇, wherein R₆ and R₇ are identical or different and denote hydrogen or (C₁-C₁₀)-alkyl; —NH—CH(CONH₂)—(CH₂)₄—NH—CO—Y-PEG_{5-60K},
wherein Y is oxygen or an NH group, or NH—R₈-Z-PEG_{5-60K}, wherein R₈ denotes a chemical bond or a genetically coded amino acid from the group S, C, K or R, and
Z denotes a spacer by way of which PEG is linked;
   and
   (ii) at least one other compound wherein the other compound activates the reperfusion injury salvage kinase pathway or inhibits the mitochondrial permeability transition pore.

In one aspect of the invention, there is provided methods for treating or preventing ischemia/reperfusion injury comprising administering to a patient in need thereof an effective amount of any of the aforementioned compositions alone or in combination.

In another aspect of the invention, there is provided methods for treating or preventing an inflammatory disease or disorder comprising administering to a patient in need thereof an effective amount of any of the aforementioned compositions alone or in combination.

In yet another aspect of the invention, there is provided methods for treating or preventing vascular leak comprising administering to a patient in need thereof an effective amount of any of the aforementioned compositions alone or in combination.

In still another aspect of the invention, there is provided kits comprising the composition of any of the aforementioned compositions alone or in combination and instructions for use thereof.

The compositions described herein comprise: (i) at least one component that binds VE-cadherin and (ii) at least one other component that activates the reperfusion injury salvage kinase (RISK) pathway or inhibits the opening of the mitochondrial permeability transition pore (MPTP).

### DETAILED DESCRIPTION OF THE INVENTION

### A. Pharmaceutical Compositions

The compositions may be selected from

H₂N-GHRPX₁X₂X₃-β-X₄X₅X₆X₇X₈X₉X₁₀-X₁₁ (I),

in which
X₁-X₁₀ denote one of the 20 genetically coded amino acids, wherein X₂, X₃, X₆, X₇, X₈, X₉ and X₁₀ individually or jointly may also denote a single chemical bond
X₁₁ denotes OR₁ in which R₁ equals hydrogen or (C₁-C₁₀) alkyl,
   NR₂R₃ with R₂ and R₃ are equal or different and denote hydrogen, (C₁-C₁₀) alkyl or a residue
   —W-PEG_{5-60K}, in which the PEG residue is attached via a suitable spacer W to the N-atom, or
   a residue
   NH—Y-Z-PEG_{5-60K},
   in which
   Y denotes a single chemical bond or a genetically coded amino acid from the group S, C, K or R and in which
   Z denotes a spacer, via which a polyethylene glycol (PEG)-residue can be attached, as well as their physiologically acceptable salts,
   and in which additionally
β denotes an amino acid, whether genetically coded or not, or a peptidomimetic element, which have the additional property of inducing a bend or turn in the peptide backbone.

Such amino acids include without limitation L-proline, D-proline, L-hydroxyproline, D-hydroxyproline, L-(O-benzyl)-hydroxyproline, D-(O-benzyl)-hydroxyproline, L-(O-tert. butyl)-hydroxyproline, 4-(O-2-naphtyl)-hydroxyproline, naphtyl-methyl)-hydroxyproline, 4-(O-phenyl)-hydroxyproline, 4-(4-phenyl-benzyl)-proline, cis-3-phenyl-proline, cis-4-phenyl-proline, trans-4-phenyl-proline, cis-5-phenyl-proline, trans-5-phenyl-proline, 4-benzyl-proline, 4-bromobenzyl-proline, 4-cyclohexyl-proline, 4-fluor-proline, L-tetrahydroisoquinoline-2-carboxylic acid (L-Tic), all diastereomers of octahydro-indole-2-carboxylic acid (Oic), and all diastereomers of 1-aza-bicyclo[3,3,0]octane-2-carboxylic acid. Additional amino acids having the turn-inducing property are know to one skilled in the art and are compounds of formula I containing them also subject of this invention.

Peptidomimetic elements pertaining to this invention are residues, which are able to replace one or several amino acids of a peptide chain and which also have the additional property of inducing a bend or turn in the peptide backbone. Several such residues have for instance been described in the patent application WO2005/056577, in which they were used for the preparation of peptidic HIV inhibitors.

A selection of useful peptidomimetic elements for the purpose of this invention are, without limitation the following:

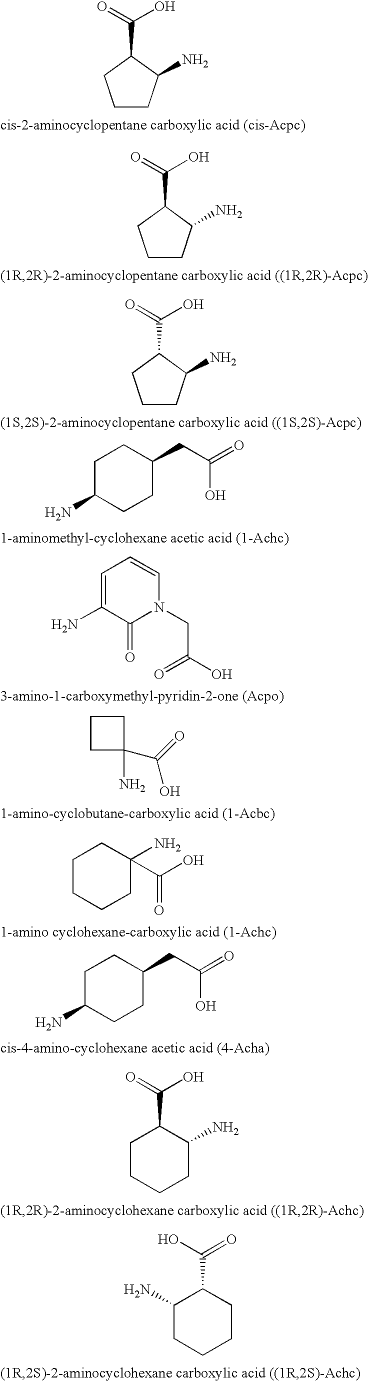

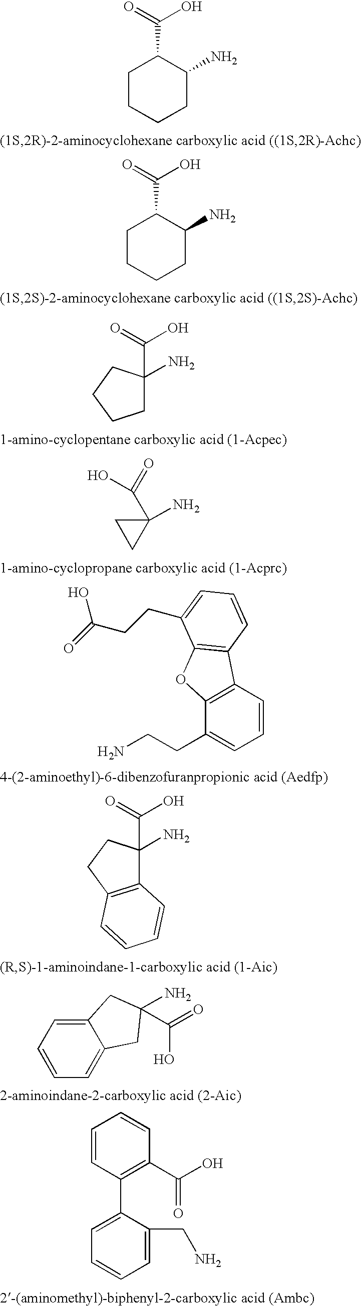

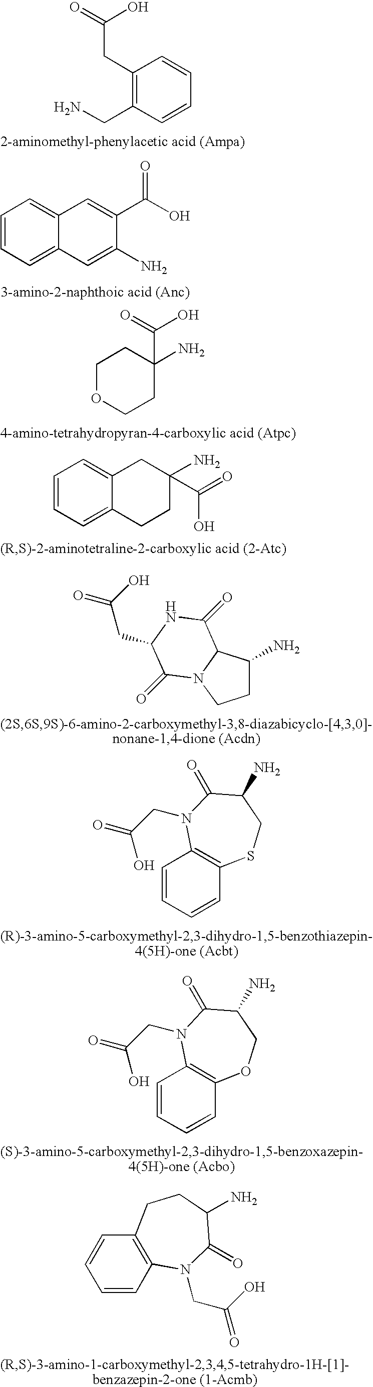

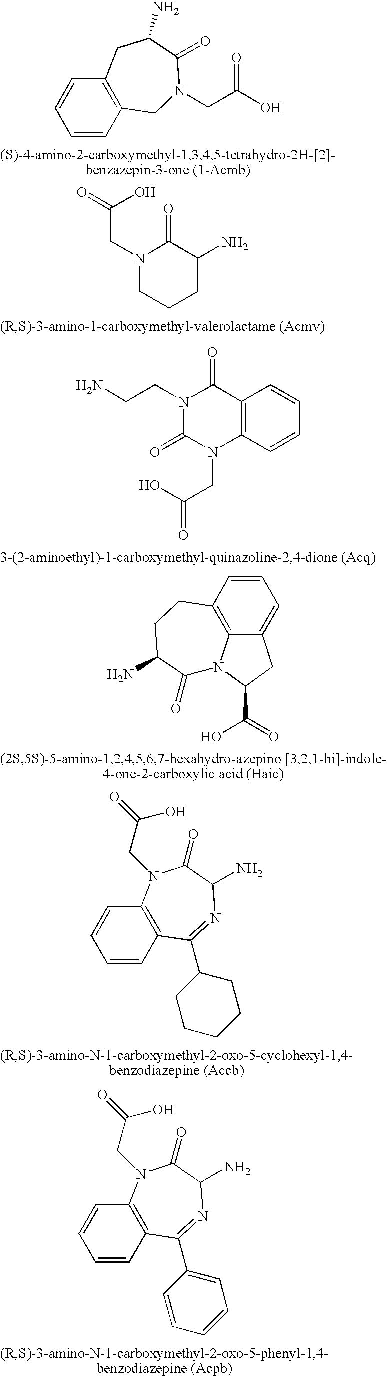

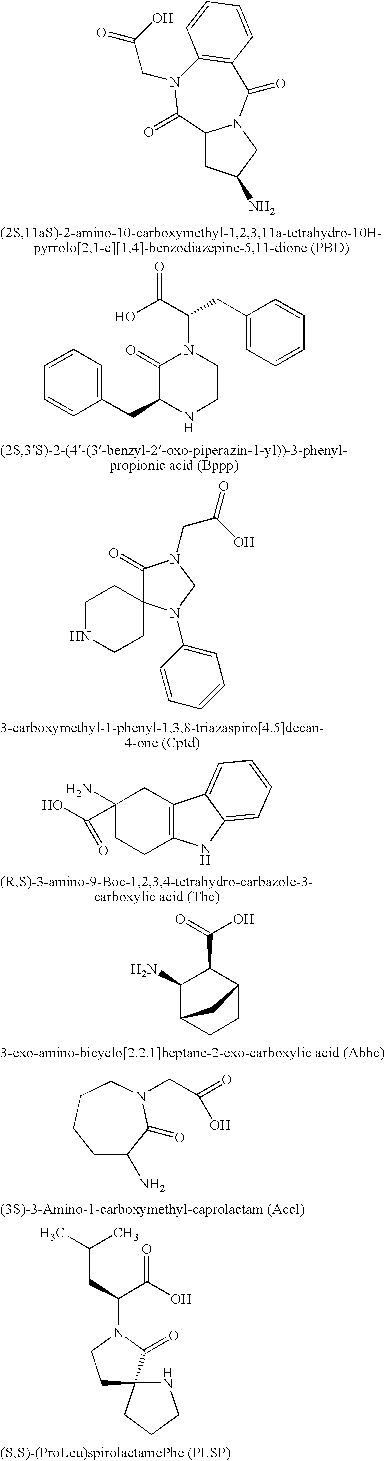

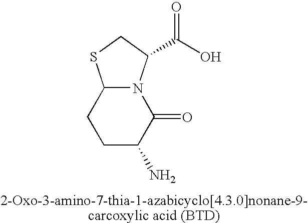

Cyclophilin D, located in the matrix of mitochondria, is a component of the mitochondrial permeability transition pore. The pore opening raises the permeability of the mitochondrial inner membrane, allows influx of cytosolic molecules into the mitochondrial matrix, increases the matrix volume, and disrupts the mitochondrial outer membrane.

Compounds that bind to cyclophilin D or compounds that inhibit the opening of the MPTP directly or indirectly including, but not limited to, Group A: cyclosporine, sanglifehrin A, NIM811, atrial natriuretic peptide, atorvastatin, glucagon-like peptide-1, exendin-4, erythropoietin, and darbapoietin, among others.

In one embodiment said pharmaceutical preparation contains a peptide with the following sequence:

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg

and a compound from group A.

In another preferred embodiment, the pharmaceutical composition contains a VE-cadherin binding compound selected from WO 02/48180, WO 07/95659, WO 07/95660, WO 07/95661 or a compound of general formula (I).

Exemplary compounds from WO 02/48180 include those referred to therein as formulas I, II, as well as preferred embodiments thereof, detailed on pages 2, 3, 5, 6, and 8-9 therein (corresponding to US Patent publication 2004/0192596 of U.S. Ser. No. 10/459,030 paragraphs [0002]-[0017], [0019]-[0023], [0025] and [0026]), which are reproduced below.

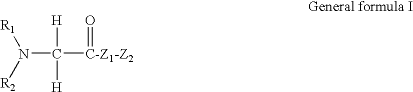

wherein R₁ and R₂, being equal or different, denote hydrogen, a saturated or unsaturated hydrocarbon residue comprising from 1 to 3, in particular up to 10, carbon atoms,
Z₁ denotes a histidine or proline residue,
Z₂ denotes an arginine residue, a peptide residue or a protein residue comprising an initial arginine residue, in particular comprising from 2 to 30 amino acids, as well as the salts thereof, and, f.i., also amides, or mixtures with each other and/or with at least one further substance for therapeutic and/or preventive use in human and/or veterinary medicine, whereby in particular only L-amino acids are provided.

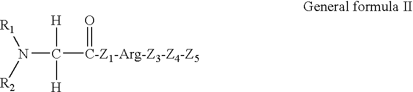

wherein R₁ and R₂, being equal or different, denote hydrogen, a saturated or unsaturated hydrocarbon residue comprising from 1 to 3, in particular up to 10, carbon atoms,
Z₁ denotes a histidine or proline residue,
Arg denotes an arginine,
Z₃ denotes a proline or valine residue,
Z₄ denotes a leucine or valine residue,
Z₅ denotes a protein residue or a peptide residue, in particular comprising from 2 to 30 amino acids, or an alcohol comprising from 1 to 3, in particular up to 10, carbon atoms, or an organic or inorganic base residue, as well as the salts thereof, and, f.i., also amides, or mixtures with each other and/or with at least one further substance for therapeutic and/or preventive use in human and/or veterinary medicine, whereby in particular only L-amino acids are provided.

A peptide or protein according to the invention of the general formula II, wherein Z₅ denotes a peptide residue comprising the following amino acid sequence:

Asp Lys Lys Arg Glu Glu Ala Pro Ser Leu Arg Pro
Ala Pro Pro Pro Ile Ser Gly Gly Gly Tyr Arg

and Z₁ denotes a histidine residue,
Arg denotes an arginine residue,
Z₃ denotes a proline residue,
Z₄ denotes a leucine residue

A peptide or protein of the general formula II, wherein Z₅ denotes a peptide residue comprising the following amino acid sequence:

Glu Arg His Gln Ser Ala Cys Lys Asp Ser Asp Trp
Pro Phe Cys Ser Asp Glu Asp Trp Asn Tyr Lys

and Z₁ denotes a proline residue,
Arg denotes an arginine residue,
Z₃ denotes a valine residue,
Z₄ denotes a valine residue

Peptide Aalpha corresponds to amino acids 1 to 28 of the alpha chain of the fibrin and is identical to amino acids 17 to 45 of the Aalpha chain of the fibrinogen:

Gly Pro Arg Val Val Glu Arg His Gln Ser Ala Cys
Lys Asp Ser Asp Trp Pro Phe Cys Ser Asp Glu Asp
Trp Asn Tyr Lys

Peptide Bbeta corresponds to amino acids 1 to 28 of the beta chain of the fibrin and is identical to amino acids 15 to 43 of the Bbeta chain of the fibrinogen, which exhibits the following sequence:

Gly His Arg Pro Leu Asp Lys Lys Arg Glu Glu Ala
Pro Ser Leu Arg Pro Ala Pro Pro Pro Ile Ser Gly
Gly Gly Tyr Arg

Exemplary compounds from WO 07/95660 include those referred to therein as formulas I, II, III, as well as preferred embodiments thereof, detailed on pages 4-7 therein, which are reproduced below.

H₂N-GHRPX₁X₂X₃X₄X₅X₆X₇X₈PX₉X₁₀X₁₁PX₁₂PPPX₁₃X₁₄X₁₅X₁₆GYR-X₁₇, (I)

wherein:
X₁-X₁₆ denote one of the 20 genetically encoded amino acids,
X₁₇ denotes OR₁ with R₁=hydrogen or (C₁-C₁₀)-alkyl, or NR₂R₃, R₂ and R₃ being identical or different and denoting hydrogen, (C₁-C₁₀)-alkyl, or a residue —PEG_{5-60K}, wherein the PEG-residue is linked to the N atom via a spacer, or a residue NH—Y-Z-PEG_{5-60K}, wherein Y denotes a chemical bond or a genetically coded amino acid from among the group of S, C, K or R, and Z denotes a spacer by way of which a polyethylene glycol (PEG)-residue may be linked, as well as the physiologically acceptable salts thereof,
   or wherein
X₁₅ or X₁₆ denote an amino acid from the group of C or K, which is linked to a residue Z-PEG_{5-60K} via the heteroatom in the side chain, and wherein
X₁₇ denotes OR₁, with R₁=hydrogen or (C₁-C₁₀)-alkyl, or NR₂R₃, R₂ and R₃ being identical or different and denoting hydrogen or (C₁-C₁₀)alkyl,
   as well as the physiologically acceptable salts thereof.

A preferred subject matter of the invention are peptides and peptide derivatives of the general Formula I, wherein:

X₁, X₉, X₁₀, X₁₄ denote L, I, S, M or A,
X₂, X₆, X₇ denote E or D,
X₃, X₄, X₅, X₁₁ denote R or K
X₈, X₁₂ denote A, G, S, or L
X₁₃ denotes I, L or V and wherein
X₁₅, X₁₆ and X₁₇ have the same meaning as given above,
as well as the physiologically acceptable salts thereof.

A particularly preferred subject matter of the invention are peptides and peptide derivates of Formula II,

H₂N-GHRPLDKKREEAPSLRPAPPPISGGGYR-X₁₇ (H),

wherein X₁₇ has the same meaning as given above for Formula I, as well as the physiologically acceptable salts thereof.

A most highly preferred subject matter of the present invention are compounds of Formula (II), wherein
X₁₇ denotes NR₂R₃, R₂ and R₃ being identical or different and being hydrogen or (C₁-C₁₀)-alkyl, or a residue C(NR₂R₃)—(S-succinimido)-(PEG_{5-40K}), the succinimide residue being linked via C-atom 3 to the sulfur atom of the cysteine residue,
   as well as the physiologically acceptable salts thereof.

A furthermore most highly preferred subject matter of the invention are peptide derivatives of Formula (III),

H₂N-GHRPLDKKREEAPSLRPAPPPIS-X₁₉-X₂₀-X₂₁-YR-X₁₇ (III)

wherein two of the residues X₁₉, X₂₀ and X₂₁ each are a glycine residue and the remaining one is a residue C—(S-succinimido)-(PEG_{5-40K}), the succinimido residue being linked to the sulfur atom of the cysteine residue via C-atom 3, and wherein X₁₇ denotes NR₂R₃, R₂ and R₃ being identical or different and being hydrogen or (C₁-C₁₀)-alkyl,
as well as the physiologically acceptable salts thereof.

A furthermore most highly preferred subject matter of the invention are peptide derivatives of Formula (III),

H₂N-GHRPLDKKREEAPSLRPAPPPIS-X₁₉-X₂₀-X₂₁-YR-X₁₇ (III)

wherein two of the residues X₁₉, X₂₀ and X₂₁ each are a glycine residue and the remaining one is a residue K-(PEG_{5-40K}), the PEG-residue being linked via the nitrogen atom in the side chain of the lysine residue, and wherein
X₁₇ denotes NR₂R₃, R₂ and R₃ being identical or different and being hydrogen or (C₁-C₁₀)-alkyl, as well as the physiologically acceptable salts thereof.

Exemplary compounds from WO 07/95661 include those referred to therein as formulas Ia, Ib, IIa, IIb, and preferred embodiments thereof, detailed on pages 4-7 therein, which are reproduced below.

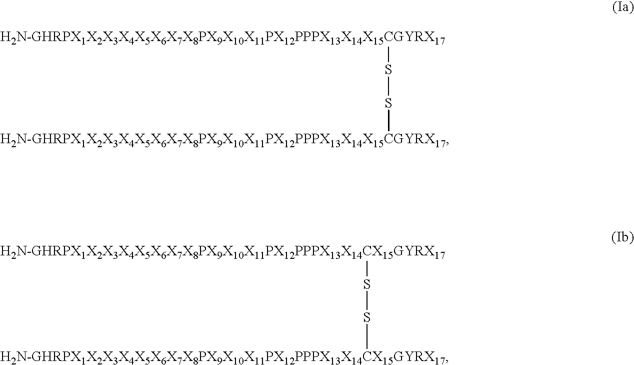

wherein:
X₁-X₁₅ denote one of the 20 genetically encoded amino acids,
X₁₇ denotes a residue OR₁, wherein R₁=hydrogen or (C₁-C₁₀-alkyl),
or a residue NR₂R₃, R₂ and R₃ being identical or different and hydrogen or (C₁-C₁₀)-alkyl,
or a residue -PEG_{5-60K}-CO—NR₄R₅, R₄ and R₅ being identical or different and hydrogen, (C₁-C₁₀)-alkyl,
or a residue —NH—CH(CONH₂)—(CH₂)₄—NH—CO—Y-PEG_{5-60K},
wherein Y may in turn be an oxygen atom or an NH group,
or a residue NH—Y-Z-PEG_{5-60K}, wherein Y denotes a chemical bond or a genetically coded amino acid from the group S, C, K or R, and Z denotes a spacer by way of which a polyethylene glycol (PEG)-residue is linked, as well as the physiologically acceptable salts thereof.

A preferred subject matter of the invention are peptides and peptide derivates of the general Formula I, wherein:
X₁, X₉, X₁₀, X₁₄ denote L, I, S, M or A,
X₂, X₆, X₇ denote E or D,
X₃, X₄, X₅, X₁₁ denote R or K
X₈, X₁₂ denote A, G, S, or L
X₁₃ denotes I, L or V
X₁₅ denote G, A, S or C
and wherein X₁₇ is as defined above, as well as the physiologically acceptable salts thereof.

A particularly preferred subject matter of the invention are peptides and peptide derivatives of Formulas IIa and IIb

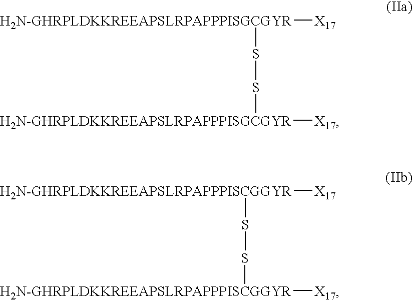

wherein X₁₇ is as defined above for Formula I, as well as the physiologically acceptable salts thereof.

A most highly preferred subject matter of the invention are compounds of Formulas (IIa) and (IIb), wherein X₁₇ denotes NH₂ or NR₂R₃, R₂ and R₃ being identical or different and hydrogen, or (C₁-C₃)-alkyl, or a residue -PEG_{5-30K}-CO—NR₄R₅, R₄ and R₅ being the same or different and hydrogen or (C₁-C₃)-alkyl, or a residue NH—CH(CONH₂)—(CH₂)₄—NH—CO—Y-PEG_{5-30K}, wherein Y may be an oxygen atom or an NH group or a residue C(NR₂R₃)—(S-succinimido)-(PEG_{5-40K}), the succinimide residue being linked via C-atom 3 to the sulfur atom of the cysteine residue, as well as the physiologically acceptable salts thereof.

In one preferred embodiment, the pharmaceutical preparation contains a peptide of the following sequence

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg

and cyclosporine.

The amino acid residues in the compounds of Formula I may either be present in their D or their L configuration.

The term peptide refers to a polymer of these amino acids, which are linked via an amide linkage.

“Physiologically acceptable” means that salts are formed with acids or bases the addition of which does not have undesirable effects when used for humans. Preferable are salts with acids or bases the use of which is listed for use with warm blooded animals, in particular humans, in the US Pharmacopoeia or any other generally recognized pharmacopoeia.

As used herein, the term “administering,” refers to any mode of transferring, delivering, introducing or transporting a pharmaceutical drug or other agent to a subject. Administering refers to said administering is intravenous, intra-arterial, subcutaneous, intramuscular, intracisternal, intraperitoneal, and intradermal, nasal (inhalation or aerosol), buccal, topical, intralesional, intracranial, intraprostatic, intrapleurally, intratracheal, intranasal, intravitreal, intravaginal, intrarectal, intratumoral, intraocular, subconjunctival, intravesicular, mucosal, intrapericardial, intraumbilical, orally, local, by injection, by infusion, by continuous infusion, by absorption, by adsorption, by immersion, by localized perfusion, via a catheter, or via a lavage.

Also contemplated by the present invention is utilization of a device or instrument in administering an agent or in an erodable implant of a suitable biologically degradable polymer (e.g., polylactate or polyglycolate). Such device may utilize active or passive transport and may be slow-release or fast-release delivery device.

The term “pharmaceutical” or “pharmaceutical drug,” as used herein refers to any pharmacological, therapeutic or active biological agent that may be administered to a subject. In certain embodiments the subject is an animal, including a vertebrate, and preferably a mammal, most preferably a human.

The term “pharmaceutically acceptable carrier,” as used herein, refers generally to any material that may accompany the pharmaceutical drug but which does not interfere with the activity of the pharmaceutical drug and which does not cause an adverse reaction with the subject's immune system.

In certain embodiments, the components are PEGylated. PEG refers to polyethylene glycol. PEGylation can significantly enhance protein half-life by shielding the polypeptide from proteolytic enzymes and increasing the apparent size of the protein, thus reducing clearance rates. Moreover, PEG conjugates can enhance protein solubility and have beneficial effects on biodistribution.

PEG can have a molecular weight of about, for example, between 0.5 Kd and 100 Kd, this molecular weight being the minimum and maximum of a molecular weight distribution, so that individual components of the mixture may have a higher or lower molecular weight. In certain embodiments, PEG has a molecular weight of about 0.5 Kd to 70 Kd. In other certain embodiments, PEG has a molecular weight of about 0.5 Kd to 60 Kd. In other certain embodiments, PEG has a molecular weight of about 0.5 Kd to 40 Kd. In further other certain embodiments, PEG has a molecular weight of about 5 Kd to 30 Kd. PEG may be linear or branched.

### B. Methods of Preparing Liquid Pharmaceutical Compositions

The pharmaceutical preparations according to this invention may be formulated together with pharmaceutical adjuvants and additives. Preparation of such formulations include a therapeutically effective dose of the pharmacologically active components of the composition is mixed with pharmaceutically acceptable diluents, stabilizers, solubilizers, emulsifying aids, adjuvants or carriers and brought into a suitable therapeutic form. Such preparations for instance contain a dilution of various buffers (e.g., Tris-HCl, acetate, phosphate) of different pH and ionic strength, detergents and solubilizers (e.g., Tween 80, Polysorbat 80), antioxidants (e.g., ascorbic acid), and fillers (e.g., lactose, mannitol) (see: The United States Pharmacopeia—National Formulary 29^{th} Edition, (2006) Rockville, Md.; Remington's Pharmaceutical Sciences (2005) 21^{st} Edition, Troy, D B, Ed. Lippincott, Williams and Wilkins).

The pharmaceutical preparation may contain concentrations of the active substances that will lead to doses in a range of 0.001 to 500 mg/kg of each component, preferentially in a range of 0.1 to 100 mg/kg mg, 0.1 to 10 mg/kg mg, or mg/m2, or any range derivable therein.

### C. Methods of Using the Pharmaceutical Composition

The term “biological matter” refers to any living biological material, including cells, tissues, organs, and/or organisms, and any combination thereof. It is contemplated that the methods of the present invention may be practiced on a part of an organism (such as in cells, in tissue, and/or in one or more organs), whether that part remains within the organism or is removed from the organism, or on the whole organism. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that the methods of the present invention may be practiced on in vivo and/or isolated biological matter.

The terms “tissue” and “organ” are used according to their ordinary and plain meanings. In certain embodiments, the tissue or organ is “isolated,” meaning that it is not located within an organism.

The terms “hypoxia” and “hypoxic” refer to an environment with levels of oxygen below normal. Hypoxia occurs when the normal physiologic levels of oxygen are not supplied to a cell, tissue, or organ. “Normoxia” refers to normal physiologic levels of oxygen for the particular cell type, cell state or tissue in question. “Anoxia” is the absence of oxygen. “Hypoxic conditions” are those leading to cellular, organ or organismal hypoxia.

In one embodiment, the term “effective amount” refers to the amount that can achieve a measurable result.

Compositions maybe administered by various methods including without limitation: intravenous, intra-arterial, subcutaneous, intramuscular, intracisternal, intraperitoneal, intradermal, nasal (inhalation or aerosol), buccal, topical, intralesional, intracranial, intraprostatic, intrapleurally, intratracheal, intranasal, intravitreal, intravaginal, intrarectal, intratumoral, intraocular, subconjunctival, intravesicular, mucosal, intrapericardial, intraumbilical, orally, local, by injection, by infusion, by continuous infusion, by absorption, by adsorption, by immersion, by localized perfusion, via a catheter, or via a lavage.

The present invention also contemplates methods for treating reperfusion injury comprises edema through vascular leak, an acute inflammation caused by penetration of activated leukocytes into tissue and cell death by necrosis and apoptosis, among other etiologies. Reperfusion injury can occur following myocardial reperfusion after an acute myocardial infarction, stroke, cardiac arrest, or coronary artery bypass graft (CABG) surgery. Reperfusion injury is noted following the transplantation of an organ or following resuscitation after hemorrhagic shock or severe bleeding in traumatized patients.

The present invention contemplates methods for preventing or treatment hypoxic of ischemic injury related to transplantation of a tissue or an organ. The present invention also contemplates prevention or treatment of delayed graft function.

The present invention also contemplates methods for inducing tissue regeneration and wound healing by prevention/delay of biological processes that may result in delayed wound healing and tissue regeneration. In addition to wound healing, methods of the invention can be implemented to prevent or treat trauma such as cardiac arrest or stroke, and hemorrhagic shock. The invention has importance with respect to the risk of trauma from emergency surgical procedures, such as thoroacotomy, laparotomy, and splenic transaction or cardiac surgery, aneurysm, surgery, brain surgery and the like. The invention may be used to prevent or treat injury resulting from Systemic Inflammatory Response Syndrome (SIRS), Acute Respiratory Distress Syndrome (ARDS), kidney failure, liver failure and multi-organ failure.

In certain embodiments, methods of the present invention can be implemented to enhance survivability and prevent ischemic injury resulting from cardiac arrest or stroke comprising providing an effective amount of the composition to the patient before, after, or both before and after myocardial infarction, cardiac arrest or stroke.

In certain embodiments, methods of the present invention can be implemented to treat or prevent ischemia/reperfusion injury.

In certain embodiments, methods of the present invention can be implemented to treat or prevent an inflammatory disease or disorder.

In certain embodiments, methods of the present invention can be implemented to treat or prevent vascular leak.

The term “treatment of a disease” as used herein refers to the management and care of a patient having developed the disease, condition or disorder.

In certain embodiments, methods of the present invention include pre-treating a biological material, e.g., a patient, prior to an ischemic or hypoxic injury or disease insult. These methods can be used when an injury or disease with the potential to cause ischemia or hypoxia is scheduled or elected in advance, or predicted in advance to likely occur. Examples include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples include, but are not limited to, medical conditions in which a risk of injury or disease progression is inherent (e.g., in the context of unstable angina, following angioplasty, bleeding aneurysms, hemorrhagic strokes, following major trauma, hemorrhaging or blood loss), or in which the risk can be diagnosed using a medical diagnostic test.

In certain embodiments, the amount of or effective compound that is provided to biological material can be about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 microM, mM or M, or any range derivable therein.

In various embodiments of the present invention, biological material is exposed to liquid pharmaceutical compositions of the current invention for about, at least, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

Furthermore, when administration is intravenous, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the solution is specified by volume, depending on the concentration of the liquid chalcogenide composition. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

### Example 1

### Myocardial Ischemia/Reperfusion Injury Animal Model

A myocardial ischemia/reperfusion (I/R) injury model in rats was used to examine the cardioprotective benefit of a combination treatment using peptide

Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg (II)

and cyclosporine.

Peptides were produced by solid-phase peptide synthesis and purified with reversed-phase HPLC using nucleosil 100-10C18 columns (PiChem, Graz, Austria).

As detailed below, a bolus administration of the combination of peptide II and cyclosporine intravenously post-ischemia and reperfusion reduced myocardial ischemia/reperfusion injury as demonstrated by a reduction in myocardial infarct size as a percentage of risk area.

Wistar rats with a body weight in the range of 220 to 280 g were divided into control and treatment groups. Animals received a standard diet.

Rats were aenesthetized and intubated. Artificial ventilation was initiated at a rate of 70 breaths per minute with 8-10 ml of a gas mixture of 30% oxygen. After stabilization, the left coronary artery was occluded with a suture for 30 min. After 30 min the suture was released and the myocardium reperfused. Test compounds were given intravenously at doses of 0.3 to 5 mg/kg of peptide II and 0.3 to 4 mg/kg of cyclosporine.
To differentiate damaged tissue from intact myocardial tissue, Evans' Blue was then given into the left coronary artery in a concentration of 2% w/v. The heart was then excised and cut into five horizontal slices. The slices were incubated with 15 w/v of tetraphenyl tetrazolium chloride for 20 min at 37° C. in order to differentiate between normal and infracted tissues. The slices were then analyzed using computerized planimetry.
The disruption of blood flow from the coronary occlusion leads to an area at risk of ischemic damage of approximately 64% in control rats and 61% in treated animals.
Infarct size as percent of the area at risk was shown to be 53% of area at risk in control animals and 21% in treated animals (p<0.05).

These experiments establish that the pharmaceutical compositions described herein have a protective effect on animals and provide a means of protecting and preserving biological material from hypoxic or ischemic injury.

### INCORPORATION BY REFERENCE

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A composition comprising:
(i) compound (I):
H₂N-GHRPX₁X₂X₃-β-X₄X₅X₆X₇X₈X₉X₁₀-X₁₁, or a physiologically acceptable salt thereof, wherein
X₁-X₁₀ denote one of the 20 genetically coded amino acids or wherein X₂, X₃, X₆, X₇, X₈, X₉ and X₁₀ individually or jointly denote a single chemical bond
X₁₁ denotes OR₁ wherein R₁ is hydrogen or (C₁-C₁₀) alkyl, NR₂R₃ with R₂ and R₃ identical or different and denote hydrogen, (C₁-C₁₀) alkyl; —W-PEG_{5-60K}, wherein PEG is attached via a spacer W to the N-atom of NR₂R₃; or NH—Y-Z-PEG_{5-60K}, wherein Y denotes a single chemical bond or a genetically coded amino acids from the group S, C, K or R and wherein Z denotes a spacer, via which PEG can be attached;
and
β denotes a genetically coded amino acid, a non-naturally occurring amino acid or a peptidomimetic element selected from the following: L-proline, D-proline, L-hydroxyproline, D-hydroxyproline, L-(O-benzyl)-hydroxyproline, D-(O-benzyl)-hydroxyproline, L-(O-tert. butyl)-hydroxyproline, 4-(O-2-naphtyl)-hydroxyproline, naphtyl-methyl)-hydroxyproline, 4-(O-phenyl)-hydroxyproline, 4-(4-phenyl-benzyl)-proline, cis-3-phenyl-proline, cis-4-phenyl-proline, trans-4-phenyl-proline, cis-5-phenyl-proline, trans-5-phenyl-proline, 4-benzyl-proline, 4-bromobenzyl-proline, 4-cyclohexyl-proline, 4-fluor-proline, L-tetrahydroisoquinoline-2-carboxylic acid (L-Tic), a diastereomers of octahydro-indole-2-carboxylic acid (Oic), a diastereomers of 1-aza-bicyclo[3,3,0]octane-2-carboxylic acid, 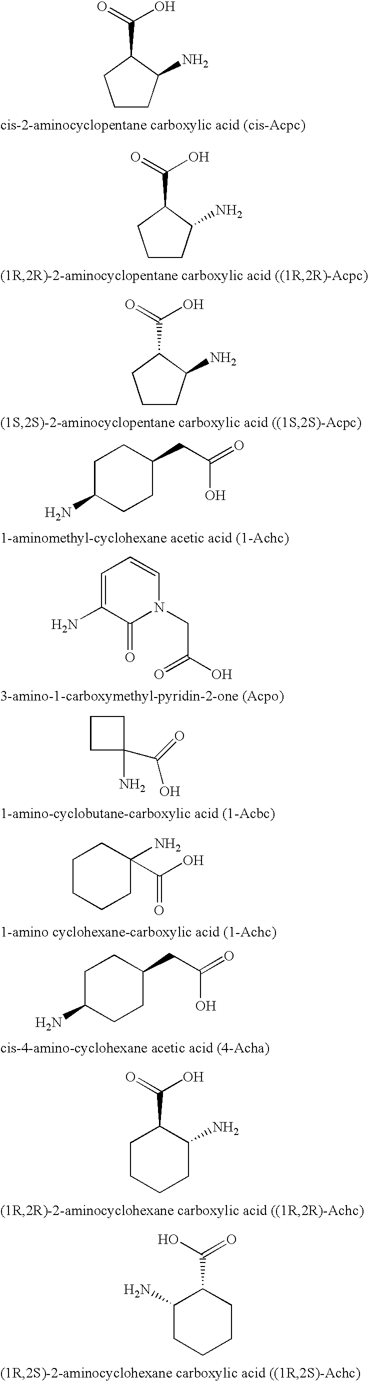 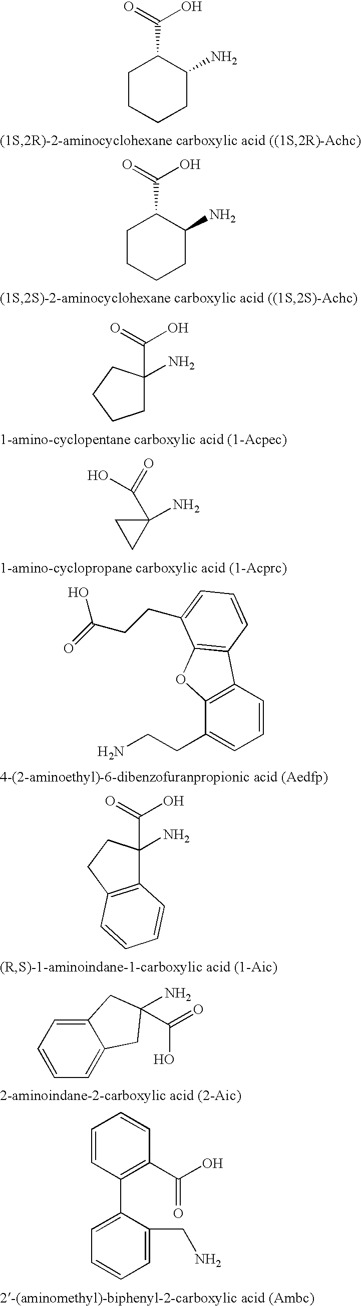 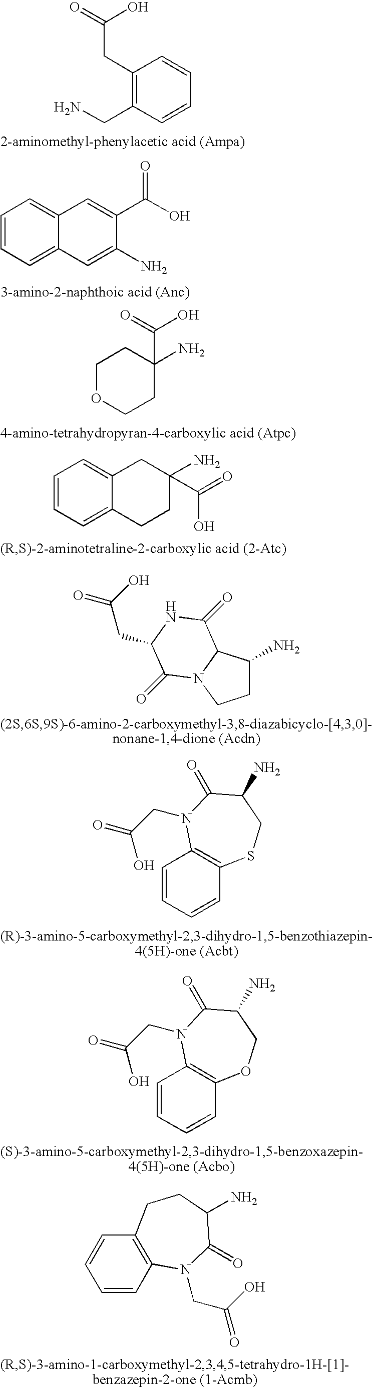 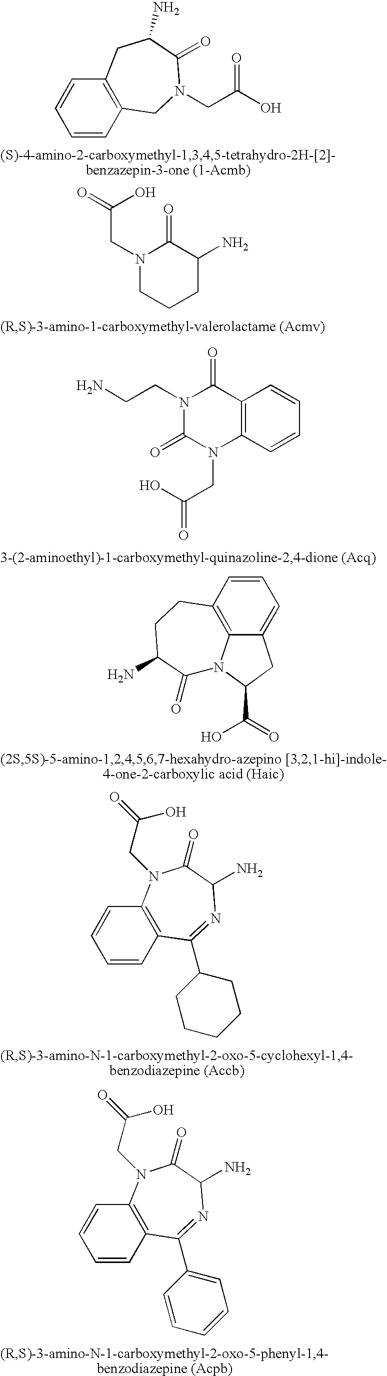 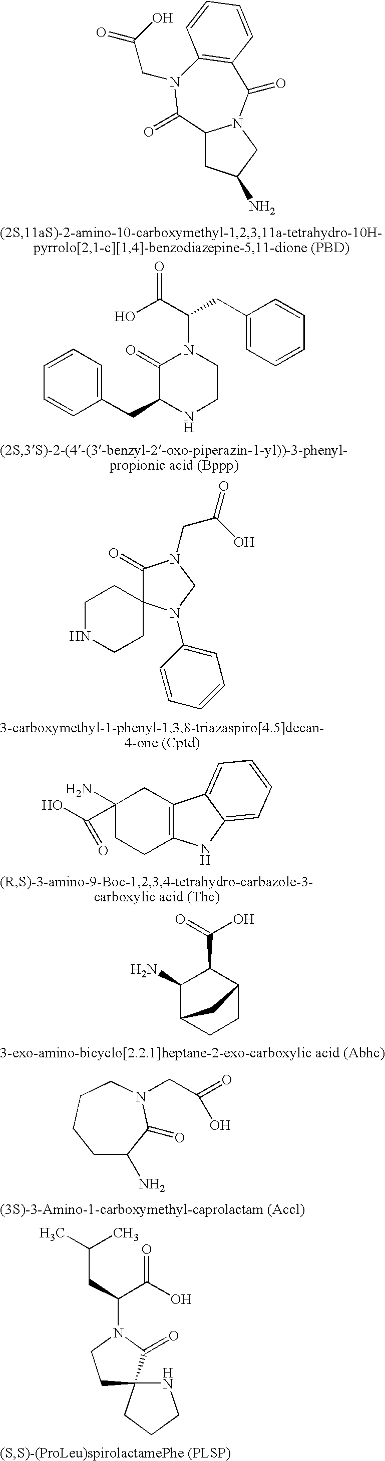 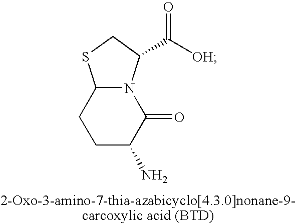
and
(ii) at least one other compound wherein the other compound activates the reperfusion injury salvage kinase pathway or inhibits the mitochondrial permeability transition pore.

2. A composition comprising:
(i) compound A
Gly-His-Arg-Pro-Leu-Asp-Lys-Lys-Arg-Glu-Glu-Ala-
Pro-Ser-Leu-Arg-Pro-Ala-Pro-Pro-Pro-Ile-Ser-Gly-
Gly-Gly-Tyr-Arg
or a salt thereof, wherein the amino terminus is 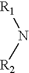
wherein R₁ and R₂ are either the same or different, and wherein R₁ and R₂ are each selected from the group consisting of hydrogen and a saturated or unsaturated hydrocarbon residue, said residue having from 1 to 10 carbon atoms;
and
(ii) at least one other compound wherein the other compound activates the reperfusion injury salvage kinase pathway or inhibits the mitochondrial permeability transition pore.

3. A composition comprising:
(i) compound B
H₂N-GHRPLDKKREEAPSLRPAPPPISGGGYR-X₁₇ or a physiologically acceptable salt thereof, wherein:
X₁₇ denotes NR₃R₄ or C(NR₃R₄)—(S-succinimido)-(polyethylene glycol (PEG)_{5-40K}), wherein the succinimide is linked to the sulfur atom of the cysteine residue via C-atom 3, R₃ and R₄ being identical or different and being hydrogen or (C₁-C₁₀)-alkyl;
and
(ii) at least one other compound wherein the other compound activates the reperfusion injury salvage kinase pathway or inhibits the mitochondrial permeability transition pore.

4. A composition comprising:
(i) at least one compound selected from:
compound C 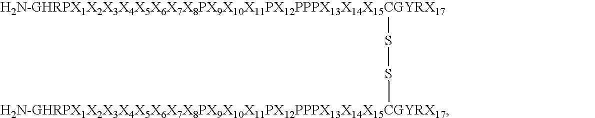
or
compound D 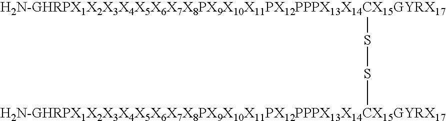
or a physiologically acceptable salt thereof, wherein:
X₁-X₁₅ denote one of the 20 genetically encoded amino acids,
X₁₇ denotes a residue OR₅, wherein R₅ is hydrogen or (C₁-C₁₀)-alkyl; NR₆R₇,
wherein R₆ and R₇ are identical or different and denote hydrogen or (C₁-C₁₀)-alkyl; —PEG_{5-60K}-CO—NR₆R₇, wherein R₆ and R₇ are identical or different and denote hydrogen or (C₁-C₁₀)-alkyl; —NH—CH(CONH₂)—(CH₂)₄—NH—CO—Y-PEG_{5-60K},
wherein Y is oxygen or an NH group, or NH—R₈-Z-PEG_{5-60K}, wherein R₈ denotes a chemical bond or a genetically coded amino acid from the group S, C, K or R, and Z denotes a spacer by way of which PEG is linked;
and
(ii) at least one other compound wherein the other compound activates the reperfusion injury salvage kinase pathway or inhibits the mitochondrial permeability transition pore.

5. The composition of claim 2, comprising compound A or a salt thereof, wherein R₁ and R₂ are both hydrogen.

6. The composition of claim 3, comprising compound B or a physiologically acceptable salt thereof, wherein X₁₇ denotes NR₃R₄ or C(NR₃R₄)—(S-succinimido)-(PEG_{5-60K}), wherein the succinimide is linked to the sulfur atom of the cysteine residue via C-atom 3, R₃ and R₄ being identical and being hydrogen.

7. The composition of any one of claims 1-4, wherein at least one other compound is selected from the following: cyclosporine, sanghliferin A, NIM811, atrial natriuretic peptide, atorvastatin, glucagon-like peptide-1, exendin-4, erythropoietin, and darbapoietin.

8. The composition of any one of claims 5-6, wherein at least one other compound is cyclosporine.

9. The composition of claim 1 further comprising (iii) at least one pharmaceutically acceptable carrier.

10. The composition of claim 1, wherein at least one compound selected from (i) is PEGylated.

11. A method for treating or preventing ischemia/reperfusion injury comprising administering to a patient in need thereof an effective amount of the composition of claim 1.

12. The method of claim 11, wherein said administering is intravenous, intra-arterial, subcutaneous, intramuscular, intracisternal, intraperitoneal, intradermal, nasal (inhalation or aerosol), buccal, topical, intralesional, intracranial, intraprostatic, intrapleural, intratracheal, intranasal, intravitreal, intravaginal, intrarectal, intratumoral, intraocular, subconjunctival, intravesicular, mucosal, intrapericardial, intraumbilical, oral, local, by injection, by infusion, by continuous infusion, by absorption, by adsorption, by immersion, by localized perfusion, via a catheter, or via a lavage.

13. The method of claim 11, wherein the patient is undergoing transplantation of a tissue or an organ or wherein the patient is suffering from delayed graft function.

14. The method of claim 11, wherein the patient is being treated for one or more of the following: stroke, cardiac arrest, myocardial infarction or lung injury.

15. The method of claim 11, wherein the patient is administered the composition after experiencing an ischemic condition, a hypoxic condition or hemorrhaging.

16. The method of claim 11, wherein the composition is compound A or a salt thereof, wherein R₁ and R₂ are both hydrogen.

17. The method of claim 11, wherein the composition is compound B or a physiologically acceptable salt thereof, wherein X₁₇ denotes NR₃R₄ or C(NR₃R₄)—(S-succinimido)-(PEG_{5-60K}), wherein the succinimide is linked to the sulfur atom of the cysteine residue via C-atom 3, R₃ and R₄ being identical and being hydrogen.

18. A method for treating or preventing an inflammatory disease or disorder comprising administering to a patient in need thereof an effective amount of the composition of claim 1.

19. A method for treating or preventing vascular leak comprising administering to a patient in need thereof an effective amount of the composition of claim 1.

20. A kit comprising the composition of claim 1 and instructions for use thereof.
